(19) United States
(12) Patent Application Publication
(10) Pub. No.: US 2007/0218154 A1
(43) Pub. Date: Sep. 20, 2007

(54) New remedy: an all natural remedy for the relief of muscle and joints pain

(76) Inventor: Mary Erranna Williams, Lafayette, LA (US)

Correspondence Address: Mary Erranna Williams, Apt. 219 R, 169 South Beadle Rd., LaFayette, LA, 70508, US

(21) Appl. No.: 11/404,637

(22) Filed: Feb. 13, 2006

(51) Int. Cl. A61K 36/18

(52) U.S. Cl. 424/773

(57) ABSTRACT

New Remedy is all natural remedy for the relief of muscle and joint pain. The mode of application has been basically the same since the beginning of time—using your hands to massage a site with or without something to relieve pain. However, this product is new, and made from what was put on earth for our benefit. It is the combination of herbs and emollients that give the relief when applied.

### BACKGROUND DETAILS

I am a true believer in healing, prayers, and miracles through God's helping hands. God led me to make this medicine to help people all over the world who suffer with pain. I call it New Remedy because back in the old days, all they used was herbs, roots and leaves to relieve or heal pains, infections and injuries. I researched these herbs and put it together to make this medicine and tested it on myself, my friends and family. They were amazed at the relief they got from this product, and encouraged me to continue to do God's work.

### SPECIFICATIONS

No new matter was added. I am re-doing copies that were sent back to me for completion.

Measurements for Ingredients:

1) 2 (two) quarts Sterile water

2) 2 (two) tablespoons of each of the following fresh herbs & roots—Burdock, Hyssop, Chicken tallow tree root, Rosemary

3) 36 oz. Of Wintergreen alcohol

4) 2 (two) long stems of the Aloe vera plant

5) 1 (one) teaspoon of Oil of Myrrh

6) 15 (fifteen) Elder leaves

7) 15 (fifteen) leaves from the Chicken tallow tree

8) 1 (one) teaspoon Camphor

9) 1 (one) tablespoon Epsom salts

Process:

In a 5 quart pot, add sterile water, all herbs, roots, Aloe vera stems cut into pieces and leaves,. Bring to a rolling boil, and lower heat just enough to continue boiling for 45 minutes. Remove from heat. In a sterilized glass container put alcohol, Epsom salts, Camphor, and oils. Pour hot ingredients into the glass container over those ingredients and cover. Store in a dark place for 1 (one) week, stirring twice daily. After this process, strain out all pieces of herbs and remaining pieces. Pour into sterile containers with a spout type lid.

### COMPONENTS/INGREDIENTS

1) Sterile water

2) Wintergreen alcohol

3) Burtdock root—Arctium lappal

4) Hyssop leaves and flowers—Hyssopiem officinalis

5) Aloe vera juice

6) Chicken tallow tree root—Sapium sebiferum

7) Elder leaf—Sambucus nigra

8) Oil of Rosemary—Rosemarinus officinalis

9) Myrrh—Commiphora myrrha

10) Camphor—Camphora (white)

11) Epsom salts

12) Spikenard root—Nardostachys jatamansi

1. This remedy when applied to the area where pain persists, it penetrates and relieves the pain as it is massaged over that area. It could also be warmed and used to relieve joint pain.
